(19) United States
(12) Patent Application Publication
(10) Pub. No.: US 2025/0145833 A1
(43) Pub. Date: May 8, 2025

(54) METAL EFFECT PIGMENTS WITH SURFACE-TREATMENTS, PREPARING METHOD AND USE OF SAME

(71) Applicant: MERCK PATENT GmbH, Darmstadt (DE)

(72) Inventor: Bangyin LI, Meguro-Ku, Tokyo (JP)

(73) Assignee: MERCK PATENT GmbH, Darmstadt (DE)

(21) Appl. No.: 18/835,879

(22) PCT Filed: Feb. 6, 2023
(86) PCT No.: PCT/EP2023/052775

§ 371 Date: Aug. 5, 2024

(30) Foreign Application Priority Data
Feb. 9, 2022 (EP) 22155912.3

(51) Int. Cl. C09C 1/00; A61K 8/02; A61K 8/24; A61K 8/25; A61K 8/58; A61Q 19/00; C08K 9/02; C09D 5/36; C09D 7/40; C09D 7/62; C09D 11/037; C09D 11/50

(52) U.S. Cl. CPC C09C 1/0051; A61K 8/0258; A61K 8/24; A61K 8/25; A61K 8/585; A61Q 19/00; C08K 9/02; C09D 5/36; C09D 7/62; C09D 7/70; C09D 11/037; C09D 11/50; A61K 2800/10; A61K 2800/43; A61K 2800/612; A61K 2800/651; C09C 2200/1058; C09C 2220/106

(57) ABSTRACT

Metal effect pigments, having a surface treatment comprising A) phosphoric acid (H3PO4), B) tetraethyl orthosilicate (TEOS) and C) at least one organic coupling agent to the metal effect pigments, exhibiting excellent humidity resistance performance without any influence on the chroma, luster and color of base metal effect pigments, a process for producing same and their use.

### CROSS-REFERENCE TO RELATED APPLICATIONS

This application is a U.S. national stage application claiming priority under 35 U.S.C. §§ 120 and 365 (c) of and to PCT International Application No. PCT/EP2023/052775, filed Feb. 6, 2023, which claims priority under 35 U.S.C. §§ 119 (a) and 365 (b) of and to European Patent Application No. 22155912.3, filed Feb. 9, 2022, each of which are incorporated herein in their entirety and for all purposes.

The present invention relates to metal effect pigments with surface-treatments, the preparing method, and the use of same.

Highly anti-corrosive thin platelet-like metal pigments having high anti-corrosive properties and good dispersibility without damaging the original surface smoothness of the thin platelet-like metal substrate such as aluminum flake etc. are well known (for example, JP, A, 2003-41150). The corrosion resistance for highly anti-corrosive thin platelet-like metal substrate pigments is achieved in that after treating the surface of a thin platelet-like metal substrate with phosphoric acid compounds and/or boric acid compounds (the first layer) in non-aqueous system, thereafter a hydrated metal oxide is coated thereon (the second layer) by a sol-gel method in a non-aqueous system. Further, colored interference pigments having metallic luster are obtained by coating one or more hydrated metal oxide layers (the third layer or more) onto the outer layer of the highly anti-corrosive thin platelet-like metal pigments used as a base by a wet process method in an aqueous system (paragraph of JP, A, 2003-41150).

When effect pigments are employed in a multiplicity of different application media, for example, paints and coatings etc., quite different requirements need to be fulfilled. Primarily, the effect pigments should not only be compatible with the application medium surrounding them but must also be sufficiently stable for long periods in some cases. Surface-coating layers for outdoor applications are often subjected to extreme weather conditions and long-lasting, intense exposure to water and light over an extended time, which often results in ageing of the materials.

Effect pigments having titanium dioxide, iron oxide or other metal oxide layers sometimes show considerable changes in their properties due to the action of external influences, such as water or light. This is evident from discoloration, embrittlement and reduced mechanical and chemical stability.

To circumvent these problems, it has been proposed to subject effect pigments to an aftertreatment with the aim of improving the applicational properties. The aftertreatment frequently involves coating the effect pigments with polymers, different metal oxides/hydroxides and/or silanes. For example, WO 99/57204 describes the use of reactive surface modifiers for the preparation of effect pigments which exhibit good orientation and distribution in surface coatings and WO 94/01498 describes the use of three layers of metal oxides applied on top of each other. WO 98/13426, DE 103 48 174 and U.S. Pat. No. 4,544,415 describe pigments with layers comprising metal oxides and monomeric, oligomeric, and polymeric coupling agents. For example, it is possible to carry out treatments for light resistance, water resistance and weather resistance required for application as automobile paints (for example, according to JP, A, 63-130673, JP, A, 01-292067, etc.), e.g. treatments for high plane orientation properties (leafing) required e.g. in the painting and printing fields (for example, according to JP, A, 2001-106937, JP, A, 11-347084), water-borne treatments for water-borne paints or inks (for example, according to JP, A, 8-283604), silicon treatment for improving dispersibility and hydrogenpolysiloxane treatment for improving hydrophobic and oleophobic properties for applications in the cosmetics field, surface treatments for weld-line prevention when used as resin (for example, according to JP, A, 03-100068, and JP, A, 03-93862), and different treatments for improving dispersibility. However, the effect pigments according to the state of the art are disadvantageous with respect to photostability and/or compatibility with the application medium. What is more, none of the above additional surface treatments confer the metal effect pigments with considerable improvement in their humidity resistance performance. In other words, metal effect pigments without surface treatment or only with insufficient surface treatment show decreased appearance quality over time and are unstable against humidity resistance in customer waterborne systems.

### SUMMARY OF THE INVENTION

Thus, there continues to be a demand for metal effect pigments with improved humidity resistance performance without any influence on the chroma, luster and color of the base metal effect pigments and a simple preparation of such metal effect pigments.

Accordingly, an object of the present invention is to provide metal effect pigments with surface-treatments which can achieve good humidity resistance performance which is important for outdoor uses without any influence on the chroma, luster and color of the base metal effect pigments.

To solve the above problems, the inventors of the present invention have carried out eager investigations and found that performing surface treatments with A) phosphoric acid (H₃PO₄), B) tetraethyl orthosilicate (TEOS) and C) at least one organic coupling agent to the substrate of the metal effect pigments can surprisingly improve the humidity resistance performance of the metal effect pigments. Furthermore, the present invention provides a possibility to stabilize metal effect pigments with a surface treatment that does not influence chroma, luster and color of base metal effect pigments.

Thus, the present invention relates to metal effect pigments, which have a surface treatment with A) phosphoric acid (H₃PO₄), B) tetraethyl orthosilicate (TEOS) and C) at least one, preferably two or more, organic coupling agents. In a very preferred embodiment, three organic coupling agents are used.

The present invention also relates to the above-mentioned metal effect pigments, wherein at least three organic coupling agents are used.

The present invention also relates to the above-mentioned metal effect pigments, wherein the organic coupling agent(s) is (are) selected from alkyl silanes, epoxy silanes, (meth)acryl silanes, amino silanes, and vinyl silanes.

The present invention further relates to the above-mentioned metal effect pigments, wherein the amount of C) organic coupling agent/agents is in the range of 0.3-3.0% by weight calculated as carbon content based on the total weight of the metal effect pigments.

The present invention further relates to the above-mentioned metal effect pigments, wherein the amount of A) phosphoric acid compounds (H₃PO₄) is in the range of 0.01-1.0% by weight calculated as P₂O₅ based on the total weight of the metal effect pigments.

The present invention further relates to the above-mentioned metal effect pigments, wherein the amount of B) tetraethyl orthosilicate (TEOS) is in the range of 0.1-1.5% by weight calculated as SiO₂ based on the total weight of the metal effect pigments.

The present invention further relates to the above-mentioned metal effect pigments, wherein the metal effect pigments are comprising a thin platelet-like metal substrate, coated with one or more layers of metal compounds selected from metal oxides, metal oxide hydrates, metal suboxides, metals, metal fluorides, metal sulfides, metal carbides, metal nitrides, metal oxynitrides, and mixtures thereof; and wherein the thin platelet-like metal substrate is selected from at least one type of metal or metal alloy selected from aluminum, titanium, gold, silver, iron, stainless steel, copper, zinc, tin, nickel and chromium.

The present invention further relates to the above-mentioned metal effect pigments, wherein the thin platelet-like metal substrate is coated with one or more metal oxides and/or metal sulfides of one or more metals selected from the group consisting of iron, titanium, aluminum, zirconium, tin, zinc, bismuth, calcium, manganese, cerium, chromium, cobalt, silicon, and boron. In a very preferred embodiment, the thin platelet-like metal substrate is coated with one or more metal oxides selected from Fe₂O₃ and TiO₂.

The present invention further relates to the above-mentioned metal effect pigments, wherein the metal effect pigments further comprise a layer obtained from the following steps: treating the surface of the thin platelet-like metal substrate with phosphoric acids compounds and/or boric acids compounds, followed by coating one or more hydrated metal oxide layers of one or more metals selected from the group consisting of silicon, aluminum, zirconium, and titanium by a sol-gel method.

The present invention also relates to a method for preparing the above-mentioned metal effect pigments, comprising the following steps:
Dispersing/suspending the metal effect pigments in water and/or one or more solvents,
Performing the surface treatment by adding A) phosphoric acid (H₃PO₄), B) tetraethyl orthosilicate (TEOS) and C) at least one organic coupling agent into water and/or the solvent(s),
Drying the metal effect pigments.

The present invention further relates to the above-mentioned method for preparing the above-mentioned metal effect pigments, wherein the surface treatment can be performed by wet-chemical methods and/or by a sol-gel process.

The present invention also relates to waterborne coating systems, comprising the above-mentioned metal effect pigments.

The present invention also relates to the use of the above-mentioned metal effect pigments in paints, coated films, automotive coatings, industrial coatings, pigment preparations, pigment pastes, radar transparent coatings, lidar applications, painted materials, inks, printed materials, plastics, moldings, laser marking, or cosmetics.

The metal effect pigments of the present invention are comprising a thin platelet-like metal substrate, coated with one or more layers of metal compounds selected from metal oxides, metal oxide hydrates, metal suboxides, metals, metal fluorides, metal sulfides, metal carbides, metal nitrides, metal oxynitrides, and mixtures thereof. Based on the metal effect pigments, the surface treatments comprising A) phosphoric acid (H₃PO₄), B) tetraethyl orthosilicate (TEOS) and C) at least one organic coupling agent to the substrate are performed.

The metal effect pigments with the surface-treatments show excellent humidity resistance performance and compatibility with the application medium compared with a case with no surface-treatment, or only with insufficient surface-treatments, i.e., a surface treatment only with e.g., the organic coupling agent(s). Furthermore, the present invention provides a possibility to stabilize metal effect pigments that do not influence chroma, luster and color of base metal effect pigments.

Moreover, since the surface-treatments can be conducted by utilizing the so-called wet-process method, the procedure is simple and easy to operate, meanwhile the cost for manufacturing is lowered.

Hereinafter, the present invention will be explained in more detail together with the preparing method.

### DETAILED DESCRIPTION OF THE INVENTION

The thin platelet-like metal substrate used in the present invention can be selected from at least one type of metal or metal alloy selected from aluminum, titanium, gold, silver, iron, stainless steel, copper, zinc, tin, nickel, and chromium. On top of the thin platelet-like metal substrate, one or more layers of metal compounds selected from metal oxides, metal oxide hydrates, metal suboxides, metals, metal fluorides, metal sulfides, metal carbides, metal nitrides, metal oxynitrides, and mixtures thereof can be coated. For example, the thin platelet-like metal substrate can be coated with one or more metal oxides and/or metal sulfides of one or more metals selected from the group consisting of iron, titanium, aluminum, zirconium, tin, zinc, bismuth, calcium, manganese, cerium, chromium, cobalt, silicon, and boron.

The highly anti-corrosive thin platelet-like metal substrate used in the present invention has a layer formed by an anti-corrosive treatment in a non-aqueous system. The thin platelet-like metal substrates as the core of the above-mentioned highly anti-corrosive thin platelet-like metal substrates are composed of metals and metal alloys.

The thin platelet-like metal substrates used in the present invention have an average particle diameter of 2 to 100 μm and an average thickness of 0.02 to 5 μm, preferably an average particle diameter of 5 to 50 μm and an average thickness of 0.02 to 2 μm, and more preferably, they have an average particle diameter of 5 to 30 μm and an average thickness of 0.05 to 1 μm. Specific examples of flakes include aluminum flakes, titanium flakes, iron flakes, bronze flakes, stainless steel flakes, aluminum bronze flakes, various aluminum alloy flakes, various titanium alloy flakes, and others. Preferred flakes include aluminum flakes, titanium flakes, stainless steel flakes, bronze flakes, etc.; and even more preferred flakes include aluminum flakes (for example, supplied by Silver Line Co. Ltd., Showa Aluminum Co. Ltd., Toyo Aluminum Co. Ltd., Asahi-Kasei Metals Co. Ltd., Eckart-Werke, etc.), titanium flakes, and stainless-steel flakes, etc. of which there is a stable supply commercially available as the brilliant metallic pigments. In particular aluminum flakes are preferred having an average particle diameter of 5 to 50 μm.

Among these, thin platelet-like metal substrates commercially available in various states may be used, such as those substrates that have already been suspended in an organic solvent to prevent the oxidative corrosion caused by the moisture contained in the air (for example, pigment pastes suspended in mineral spirit etc.), those substrates that, for the purpose of leafing or for improving dispersibility, have been treated with different types of surface treatment agents and have been suspended in an organic solvent and those substrates on whose surface an oxidation protection film (passivation film, i.e. surface oxidized thin-film layer) has been applied beforehand. Regarding the object of the present invention, its effect is brought out particularly by metal flakes having high corrosiveness, as long as the surface is largely free from oxidation, hence the use thereof is preferred. For example, those substrates with high corrosiveness such as aluminum flakes which are available in the market in the state of a suspension in an organic solvent before being handled and those substrates which have been treated with different surface treatment agents and have been suspended in an organic solvent are particularly recommended for use in the present invention. It is also possible to use thin platelet-like metal substrates and thin platelet-like alloy substrates which have been subject beforehand to an anti-corrosive (passivation) treatment.

The metal substrates having a layer formed by an anti-corrosive treatment in a non-aqueous system used in the present invention include thin platelet-like metal substrates which are treated on their surface with phosphoric acid compounds and/or boric acid compounds, and are further coated with one or more hydrated metal oxide layers of one or more metals selected from the group consisting of silicon, aluminum, zirconium, and titanium by sol-gel method such as, for example, described in JP, A, 2003-41150. Considering their good surface smoothness, dispersibility and surface inherent metallic luster, good adhesion of the intermediate binder layer and the hydrated iron oxide layer formed further outside thereof, the above-mentioned anti-corrosive thin platelet-like metal substrates comprising a layer of phosphoric acid compounds and/or boric acid compounds and one or more hydrated metal oxides layer of one or more metals selected from silicon, aluminum etc. by a sol-gel method are adopted.

Those treatments of highly anti-corrosive thin platelet-like metal substrates with phosphoric acid compounds and/or boric acid compounds may be carried out in accordance with the description in JP, A, 2003-41150.

The metals used for the hydrated metal oxides layer, which is to be successively formed, may be selected from the group consisting of silicon, aluminum, zirconium, and titanium. Among these, silicon and aluminum are preferred due to their good transparency and low index of refraction. Especially, silicon is preferred because it is easy to handle. The sol-gel method (described in JP, A, 2003-41150) among non-aqueous reactions for coating the second layer of this high anti-corrosive treatment is preferred to maintain the surface smoothness.

In the description of the present specification, for example, thin platelet-like metal treated with phosphoric acid compounds and/or boric acid compounds combined with further hydrated metal oxide layer in non-aqueous system described in JP, A, 2003-41150, is defined as “thin platelet-like metal substrate having a layer formed by an anti-corrosive treatment in a non-aqueous system”, thereon the layer comprising hydrated tin oxide is coated as “intermediate binder layer” (first layer), and the hydrated iron oxide layer, which is an outer layer thereof, is coated as “the second layer.” Further, the term “hydrated oxide” used in “hydrated metal oxide” generally refers to, unless otherwise stated, “oxides”, “hydroxides”, “hydrates of oxide”, and “hydrated oxides” of metal, and “mixtures thereof” in the present invention. The term “oxide” in “metal oxide” is also based on the definition for “hydrated oxide”. Moreover, when representing such “hydrated metal oxide” by chemical formula (which is described, for example, in an embodiment, etc.), it is conveniently represented by the form of oxide.

Next, the intermediate binder layer (the first layer) for improving the adhesion and denseness of the second layer will be described.

The intermediate binder layer, which is the closely followed outer layer of the above-mentioned highly anti-corrosive thin platelet-like metal substrate, can be obtained in the following way. A suitable material for the intermediate binder layer (the first layer) is hydrated tin oxide.

The above-mentioned highly anti-corrosive thin platelet-like metal substrate is dispersed in water while keeping the temperature at 60 to 90° C. to be easy to control the homogeneous coated layer, and a tin salt aqueous solution and a basic aqueous solution are simultaneously added to the suspension while keeping the pH constant thereby coating the hydrated tin oxide layer (intermediate binder layer) onto the highly anti-corrosive metal substrate. The pH is preferably less than 4.7. And more preferably, the value of pH of 0.5 to 3.0 may be adopted.

The amount of hydrated tin oxide in the intermediate binder layer (the first layer) for thin platelet-like metal substrate having a layer formed by an anti-corrosive treatment in a non-aqueous system needs to be a sufficient amount not to cause the underlying base (the layer treated in non-aqueous system) to be exposed. That is, the amount must be above the amount to be necessary to form a monolayer. The amount of hydrated tin oxide per unit area (m²) of thin platelet-like metal substrate having the layer with anti-corrosive treatment in non-aqueous system is appropriately not less than 0.0008 g as metal oxide (SnO₂).

Specifically, the amount of hydrated tin oxide used in the intermediate binder layer (the first layer) needs to be adjusted appropriately according to the type, particle size and particle size distribution of the thin platelet-like anti corrosive metal substrate. Considering the unit surface area of the highly anti-corrosive metal substrate, of course, the amount is decreased when the particle size is large, and it is necessary to increase the amount when the particle size is small.

The amount of hydrated tin oxide is adjusted to be within the possible range in which the sufficient adhesion and the improvement of denseness of the second layer are achieved and the hue of the interference color can be controlled; thus the amount per unit area (m²) of the thin platelet-like metal substrate having a layer formed by an anti-corrosive treatment in a non-aqueous system is preferably 0.0008 g to 0.3 g as metal oxide (SnO₂), more preferably 0.0009 g to 0.2 g, and further preferably 0.01 g to 0.1 g. For example, when using the highly anti-corrosive substrate (specific surface area of 3.01 m²/g (described in Table 3 of JP, A, 2003-41150)) including metal aluminum as thin platelet-like metal, the suitable amount is 0.001 g to 0.06 g per unit area (m²) of the highly anti-corrosive metal substrate.

The aqueous solution of tin salt to be used is water-soluble tin (II) salt or tin (IV) salt. For example, tin (II) chloride, tin (IV) chloride, tin (II) sulfate, tin (II) acetate, tin (II) oxalate etc. are preferred.

Hereinafter, the coating of hydrated iron oxide (the second layer) after forming the above-mentioned intermediate binder layer will be described. Though the coating of hydrated iron oxide (the second layer) onto the surface of the intermediate binder layer (the first layer) may be performed through a vapor deposition method or a sol-gel method, it is more preferable to adopt a wet process (see the description in JP, A, 2003-41150 for the definition) which, in contrast to the vapor deposition method and the sol-gel method, has no restrictions on the raw materials and production facilities, is easy to achieve a homogeneous coated layer, and is easy to operate as a simple process with a wide range of applications.

A definition of the wet process method, as preferably used in the present invention has been given before. To be more specific, in an aqueous system, the method consists of (1) in the case of neutralizing hydrolysis, selecting the desired water-soluble metal salt (for example, nitrate salt, sulfate salt, chloride, acetate salt, and further metal acid salt, etc.) and the prescribed amount of aqueous solution while separately preparing an alkaline aqueous solution (an acidic aqueous solution in the case of metal acid salt), dropping these into the suspension of highly anti-corrosive thin platelet-like metal pigment which is the base obtained beforehand while maintaining a predetermined pH to form a hydrolyzed layer on the surface, thereafter washing, filtering, drying and, if desired, calcining; and (2) in the case of thermal hydrolysis, adding the predetermined amount of the desired water-soluble metal salt to a suspension of a highly anti-corrosive thin platelet-like metal pigment which is the base obtained beforehand and by heating, forming a hydrolyzed layer and, thereafter, washing, filtering, drying and, if desired, calcining. Moreover, as a variation of the method by neutralization hydrolysis (1), a method using, instead of the alkaline aqueous solution, urea and acetoamide producing alkalinity through heating, (the so-called “homogeneous precipitation method”) can also be used.

The iron (III) salt to be used may be selected from water-soluble salts such as chloride, sulfate, and nitrate. After coating the above-mentioned intermediate binder layer (the first layer), the iron salt aqueous solution is successively added while maintaining the pH constant (not higher than 4) using an alkaline aqueous solution. Specific examples of alkaline aqueous solutions used in the present invention include aqueous alkali metal hydroxide solutions such as sodium hydroxide, potassium hydroxide etc., aqueous alkali metal carbonate solutions such as sodium carbonate, potassium carbonate etc., aqueous alkali metal bicarbonate solutions such as sodium bicarbonate, potassium bicarbonate etc., ammonium carbonate, ammonium bicarbonate or an aqueous ammonia solution etc. The amount of hydrated iron oxide is preferably 0.01 g to 1.0 g as metal oxide (Fe₂O₃) per unit area (m²) of thin platelet-like metal substrate having a layer formed by an anti-corrosive treatment in a non-aqueous system in order to achieve a reddish color and to improve its chromaticity sufficiently. Therefore, the amount may be appropriately changed depending on the hue and surface smoothness of individual thin platelet-like metal substrate, and the properties of highly anti-corrosive metal substrate obtained and treated in non-aqueous system. The temperature during this coating process is preferably the same as one during the coating of the above-mentioned intermediate binder layer (the first layer) in terms of efficiency. Thus, the obtained suspension containing the colored thin platelet-like interference pigments having metallic luster with a reddish color are then filtered and washed, dried, and calcined.

The colored interference pigments having metallic luster with a reddish color obtained as described above, exhibit a reddish body color with an interference color by coating with an intermediate binder layer to improve the adhesion and denseness of the hydrated iron oxide layer (the second layer) coated thereon.

Hereinafter, the surface treatment performed on the metal effect pigments which aims at improving their humidity resistance performance without any influence on the chroma, luster and color of base metal effect pigments according to the present invention will be described.

The surface-treatments performed on the metal effect pigments are comprising treatments with A) phosphoric acid (H₃PO₄), B) tetraethyl orthosilicate (TEOS) and C) at least one organic coupling agent.

Such surface treatments can be performed by wet-chemical methods and/or by a sol-gel process. The wet-chemical methods are easy to operate and are cost effective, while the sol-gel process renders the surface-treated pigments with better humidity resistance performance although the cost may be slightly higher.

One of the essential components of the surface treatment according to the present invention is A) phosphoric acid (H₃PO₄). After anti-corrosive treatment of the metal effect pigments with a phosphoric acid (H₃PO₄) solution, phosphate (e.g., aluminum phosphate) will be formed on the surface of the metal effect pigments.

Another one of the essential components of the surface treatment according to the present invention is B) tetraethyl orthosilicate (TEOS). After treating the metal effect pigments with the tetraethyl orthosilicate (TEOS) solution and hydrolysis, silicon hydroxide (Si(OH)₄) is formed on the surface of the metal effect pigments, which facilitates further surface treatments and makes the anchoring of organic coupling agent(s) much easier. The metal effect pigments are dried after the surface treatments. After the steps of drying, the silicon hydroxide (Si(OH)₄) becomes silicon dioxide (SiO₂).

Another one of the essential components of the surface treatment according to the present invention is C) at least one organic coupling agent. The at least one organic coupling agent is preferably anchored within the surface coating and/or to the phosphate (e.g., aluminum phosphate) from the A) phosphoric acid (H₃PO₄) treatment and/or silicon hydroxide (Si(OH)₄) from the B) tetraethyl orthosilicate (TEOS) treatment of the surface treatments and/or to the substrate, i.e., via the siloxane.

Organic coupling agents suitable and preferred for the present invention are organosilanes,-aluminates,-titanates and/or zirconates of the general formula

X₄₋ₙ₋ₘZ—Rₙ(—B—Y)ₘ
where X=OH, halogen, alkoxy, aryloxy
Z=Si, Al, Ti, Zr
R=alkyl, phenyl, or hydrogen
B=organic, at least bifunctional group (alkylene, alkyleneoxyalkylene)
Y=amino, substituted amino, hydroxyl, hydroxyalkyl, siloxane, acetoxy, isocyanate, vinyl, acryloyl, epoxide, epoxypropyloxy, imidazole or ureido group
n, m=0, 1, 2,3 where n+m<3.

The organic coupling agents are preferably compounds where Z=Si. The organic coupling agents preferably comprise alkoxysilane groups, which can be converted into corresponding hydroxyl groups by hydrolytic reaction conditions. The latter can effect anchoring via oxygen bridges. In addition, it is also possible to employ mixtures of various coupling agents, in particular a mixture of two or three, most preferably three, coupling agents, which can be applied as a mixture or individually. By appropriate choice of the coupling agents, the metal effect pigments of the invention can be matched to various application systems.

The organic coupling agent can be matched to the application medium through the choice of suitable functional groups. In addition, additional bonds to the medium can be formed via the organic coupling agent through reaction of the functional groups with corresponding functionalities in the application media. In a particular preferred embodiment, the surface of the metal effect pigments according to the invention is modified by means of a combination, matched to the application medium, of mixtures of various organic coupling agents. The hydrophobicity of the metal effect pigments surface can be matched by integration of alkyl-containing organic coupling agents, such as, for example, alkylsilanes. Besides the organosilanes, preference is also given to the use of hydrolysates and homogeneous and heterogeneous oligomers and/or polymers thereof, which can likewise be employed alone or in combination with silanes, zirconates, aluminates, zircoaluminates and/or carboxyzircoaluminates as organic coating. Particular preference is given to mixtures of various organic coupling reagents, in particular with functional groups Y which are different from one another, which ensures a particular range of applications. Especially preferred are mixtures of organosilanes, in particular mixtures comprising at least two, preferably at least three, organosilanes having different functional groups.

Examples of organosilanes are propyltrimethoxysilane, propyltriethoxysilane, isobutyltrimethoxysilane, hexyltrimethoxysilane, n-octyltrimethoxysilane, i-octyltrimethoxysilane, n-octyltriethoxysilane, n-decyltrimethoxysilane, dodecyltrimethoxysilane, hexadecyl-trimethoxysilane, vinyltrimethoxysilane. Suitable oligomeric, alcohol-free organosilane hydrolysates are, inter alia, the products marketed by Sivento under the trade name “Dynasylan®”, such as, for example, Dynasylan HS 2926, Dynasylan HS 2909, Dynasylan HS2907, Dynasylan HS 2781, Dynasylan HS 2776, Dynasylan HS 2627. In addition, oligomeric vinylsilane and also aminosilane hydrolysate are suitable as organic coating. Functionalised organosilanes are, for example, 3-aminopropyltri-methoxysilane, 3-methacryloxytrimethoxysilane, 3-methacryloxypropyl-trimethoxysilane, 3-glycidyloxypropyltrimethoxysilane, beta-(3,4-epoxycyclohexyl) ethyltrimethoxysilane, gamma-isocyanatopropyltri-methoxysilane, 1,3-bis(3-glycidoxypropyl)-1,1,3,3,-tetramethyldisiloxane, ureidopropyltriethoxysilane, preferably 3-aminopropyltrimethoxysilane, 3-methacryloxytrimethoxysilane, 3-methacryloxypropyltrimethoxysilane, 3-glycidyloxypropyl-trimethoxysilane, beta-(3,4-epoxycyclohexyl) ethyl-trimethoxysilane, gamma-isocyanatopropyltrimethoxysilane. Examples of polymeric silane systems are described in WO 98/13426 and marketed, for example, by Sivento under the trade name Hydrosil®.

Especially preferred are alkylsilanes, epoxysilanes, (meth)acrylsilanes, aminosilanes, and/or vinylsilane, especially mixtures of such silanes. Particularly preferred are hexyltrimethoxysilane, n-octyltrimethoxysilane, n-octyltriethoxysilane, n-decyltrimethoxysilane, 3-aminopropyl-trimethoxysilane, 3-methacryloxytrimethoxysilane, 3-methacryloxypropyl-trimethoxysilane, 3-glycidyloxypropyltrimethoxysilane, and/or beta-(3,4-epoxycyclohexyl) ethyltrimethoxysilane, especially mixtures of such silanes, especially mixtures of at least more than three of these silanes.

It is essential for metal effect pigments of the invention to have a carbon content of >0.3% by weight based on the total weight of metal effect pigment. The upper limit should be fixed with the quantity that starts to interfere the effect of the pigment. Generally, this is around 3.0%. Preferably, the carbon content is in the range of 0.5-3.0%, especially of 0.5-1.5%, by weight based on the total weight of metal effect pigment. The carbon content is measured by means of elemental analysis on the dry pigment, preferably by a CHN analyzer UNICUBE by Elementar.

The outer coating may not be a layered structure but a mixture of phosphate, silicon dioxide and organic compounds. Advantageously, the outer coating shows an inhomogeneous distribution of compounds, especially of oxidic compounds. If desired the outer coating may be structured in such a way that it has a multilayered structure. Preferably, the concentration of organic coupling agent(s) has a maximum at the outer surface of the metal effect pigment and decreases towards the base substrate, while a decrease of concentration of phosphate and silicon dioxide compounds starting from the inner surface of the metal effect pigment toward the outer surface is preferred. In other words, the concentration of phosphate and silicon dioxide compounds has a maximum at the inner surface of the metal effect pigment and decreases towards the outer surface of the metal effect pigment.

The treatment with A) phosphoric acid compounds is performed by adding phosphoric acid compounds to the suspension of the metal effect pigment. The amount of phosphoric acid compounds used preferably is an amount corresponding to 0.01-1.0% by weight calculated as P₂O₅, even more preferably is an amount corresponding to 0.02-0.8% by weight, and still more preferably is an amount corresponding to 0.02-0.5% by weight, based on the total weight of metal effect pigment.

After the treatment with A) phosphoric acid compounds, the treatment with B) tetraethyl orthosilicate (TEOS) is performed by adding TEOS to the suspension of the metal effect pigment. The amount of TEOS used, preferably is an amount corresponding to 0.1-1.5% by weight calculated as SiO₂, even more preferably is an amount corresponding to 0.1-1.0% by weight, and still more preferably is an amount corresponding to 0.15-0.6% by weight, based on the total weight of metal effect pigment.

All weight-% are based on the total weight of metal effect pigment.

The treatment with C) at least one organic coupling agent can be described as follows: first, the reaction for application of C) at least one organic coupling agent is preferably carried out over a period of 10 to 120 minutes, but can also be extended as desired. Then the metal effect pigment obtained is worked up and isolated by methods that are widely used by the person skilled in the art, for example by filtration, drying and sieving. The drying process can be carried out in a commercial dryer, oven or kiln, under atmosphere or in nitrogen gas or other inert gases. The drying temperature is in the range of 110 to 180° C., preferably in the range of 120 to 160° C. The drying time is in the range of 15 to 360 minutes, preferably in the range of 30 to 180 minutes, depending on the amount of material for drying.

With the surface treatments as described above, the generated phosphate (e.g., aluminum phosphate) from A) phosphoric acid (H₃PO₄), the silicon dioxide (SiO₂) from B) tetraethyl orthosilicate (TEOS) as well as the organic carbon content from C) at least one organic coupling agent possess synergistic effect to improve the humidity resistance performance. Furthermore, since mixtures of phosphate, silicon dioxide and organic compounds form very thin layer/layers on the surface of the metal effect pigment, they stabilize the metal effect pigments without influencing the chroma, luster and color of base metal effect pigments.

The present invention further relates to a pigment paste made from the above-mentioned metal effect pigments, pasted with solvents, such as mineral spirit, light aromatic solvent, Ethylene Glycol Monobutyl Ether (Butyl Cellosolve), Dibutyl phthalate (DBP), Diethylene Glycol Monoethyl Ether (Carbitol), Diethylene Glycol Monobutyl Ether (Butyl Carbitol), or Propylene Glycol Methyl Ether (PGM), preferably mineral spirit and/or Diethylene Glycol Monoethyl Ether (Carbitol).

Pigment paste with solvents results in phlegmatization of the above-mentioned metal effect pigments, which avoids dust emission during handling and transportation. Moreover, it is easy to operate when used in compositions which may additionally include further pigments, resin components and/or oil components for various applications such in paints, coated films, automotive coatings, industrial coatings, pigment preparations, pigment pastes, radar transparent coatings, lidar applications, painted materials, inks, printed materials, plastics, moldings, laser marking, or cosmetics, etc.

The metal effect pigments obtained according to the present invention may be used in compositions which may additionally include further pigments, resin components and/or oil components for various applications such as in paints, coated films, automotive coatings, industrial coatings, pigment preparations, pigment pastes, radar transparent coatings, lidar applications, painted materials, inks, printed materials, plastics, moldings, laser marking, or cosmetics, etc. The present invention also relates to painted material having at least one painted layer comprising the above-mentioned composition of resin/oil and pigment. Specific examples of them will be described below. Although not specifically mentioned, “the pigment(s) in the present invention” used in the following examples indicates “metal effect pigments” including the ones prepared by applying the above-mentioned various treatments as described in paragraph of the present application.

### Use for Paints

Examples of use in paints are organic solvent-type paints, NAD (non-aqueous dispersion) paints, water-borne paints, emulsion paints, colloidal paints and powder coating. The pigment of the present invention can be mixed in a proportion of 1 to 100 wt % with respect to the paint resins as solid parts. A proportion of 1-70 wt % is preferred. A proportion of 1-20 wt % is particularly preferable. For improving the dispersibility, the surface of the pigments in the present invention can be treated with silane coupling agents and titanium coupling agents. Examples of resin components for the paints in the present invention are acrylate resins, alkyd resins, unsaturated polyester resins, amino resins, melamine resins, polyurethane resins, epoxy resins, polyamide resins, phenol resins, cellulose resins, vinyl resins, silicone resins, fluorine resins, etc. These resins may be used alone or in combination of two or more.

In water-borne paint, emulsion-type resins comprising cross-linking resins based on acrylate melamine resin, etc. can be given as examples.

Examples of mixtures and admixtures include further pigments such as organic pigments and inorganic pigments, as well as antisagging agents, viscosity adjusting agents, sedimentation preventers, cross-linking promoters, curing agents, leveling agents, defoaming agents, plasticizers, antiseptic agents, antifungal agents, ultraviolet stabilizers, etc. Examples of further pigments used in combination with the pigments according to the present invention are titanium dioxide; calcium carbonate; clay; talc; barium sulfate; white carbon; chromium oxide; zinc oxide; zinc sulfide; zinc powder; metal powder pigments (such as aluminum flakes, colored aluminum flakes, stainless steel flakes, titanium flakes, etc.); iron black; yellow iron oxide; red iron oxide; chrome yellow; carbon black; molybdate orange; Prussian Blue; ultramarine blue; cadmium type pigments; fluorescent pigments; soluble azo dyes; insoluble azo dyes; condensed azo dyes; phthalocyanine pigments; condensed polycyclic pigments; composite oxide pigments; graphite; mica (such as muscovite, phlogopite, synthetic mica, fluorine tetra silicon mica, etc.); metal oxide coated mica (such as titanium oxide coated mica, titanium dioxide coated mica, (hydrated) iron oxide coated mica, mica coated with iron oxides and titanium oxides, mica coated with lower ordered titanium oxides); metal oxide coated graphite (such as titanium dioxide coated graphite, etc.); thin platelet-like alumina; metal oxide coated alumina (such as titanium dioxide coated alumina, iron oxide coated thin platelet-like alumina, Fe₂O₃ coated thin platelet-like alumina, Fe₃O₄ coated thin platelet-like alumina, interference color metal oxide coated thin platelet-like alumina, etc.); micaceous iron oxide (MIO); metal oxide coated MIO; metal oxide coated silica flakes and metal oxide coated glass flakes. By combining these and other pigments, novel hues can be obtained and chromaticity properties can be improved. These paints can be applied to wood, plastic, metal sheet plate, glass, ceramic, paper, film, sheets, translucent films of reflectors for LCDs, etc. Examples of uses for paints include automobiles, buildings, marine vessels, electric household appliances, canned goods, industrial equipment, traffic signs, plastics, household goods, etc.

Examples of the structure of the coated film in painted materials include, but are not limited to, e.g., a layered structure in the order of: base coat layer, middle coat layer, layer containing the pigments of the present invention and clear layer; or in the order of: base coat layer, middle coat layer containing the pigments of the present invention and clear layer, etc. Examples of the method for forming the coated film to constitute painted materials include one-coat/one-bake, two-coat/one-bake, two-coat/two bake, three-coat/one-bake, three-coat/two-bake, three-coat/three-bake, etc. Examples of the coating methods include electrostatic coating, spray coating, airless coating, roll coating, dip coating, etc.

Examples of use in radar transparent coatings, include water-borne OEM or refinish automotive coatings on plastic substrates. The plastic substrates are automotive bumpers, radiator grills, automotive back panels, door trim strips, back mirror casings or handy casings. The plastic substrates may be for example thermoplastic resins such as polycarbonate (PC), acrylonitrile-butadiene-styrene (ABS) copolymer, acrylonitrile-ethylene-styrene (AES) copolymer, and polypropylene (PP). Radar transparent coated film on a plastic substrate may preferably on an automotive bumper, made from the coating formulation containing metal effect pigments and/or the mixture with other pearlescent effect pigments. The coated film on the plastic substrate has a thickness of the dried film with 15 μm or below, wherein the percentage of weight concentration (PWC) of metal (Aluminum) part is preferably below 5% wt.

Examples of use in lidar applications include lidar transparent pigments and lidar reflective layers together within the lidar-compatible painted coating systems. The pigments of the present invention, like metallic gray colors, can be formulated with lidar reflective materials such as functional blacks to increase the lidar reflectance and detectability.

### Use for Printing Inks

Examples of use in printing inks include relief ink, lithographic printing ink, intaglio printing ink, ink for metal plates, radiation curable ink, UV ink, EB ink, flexo ink, screen ink, offset ink, gravure ink, etc. and water-borne inks thereof, etc. The pigments of the present invention can be mixed in a proportion of 1-100 wt % with respect to the resins as solid parts in the ink. A proportion of 1-70 wt % is preferred. A proportion of 1-20 wt % is particularly preferred. Moreover, the surface of the pigments in the present invention can be treated with silane coupling agents and titanium coupling agents, etc. Examples of resin components include e.g., maleic rosin resins, maleic resins, alkyd resins, polyamide resins, phenol resins, petroleum resins, urethane resins, epoxy resins, acrylate resins, butyral resins, melamine resins, epoxy resins, vinyl chloride resins, vinylidene chloride resins, cellulose resins, vinyl resins, unsaturated polyester resins, cellulose resins, etc. These resins may be used alone or in combination of two or more.

Examples of mixtures include further pigments such as organic pigments and inorganic pigments and additives such as varnishes, reducers, compounders, extra varnishes, gelling agents, drying promoters, antioxidants, anti-offsetting agents, lubricants, surface active agents, etc.

Further examples include dripping preventers, viscosity adjusting agents, sedimentation preventers, cross-linking agents, curing agents, leveling agents, defoaming agents, plasticizers, antiseptic agents, antifungal agents, ultraviolet stabilizers, etc.

Examples of further pigments used in combination with the pigments according to the present invention are extender pigments; precipitated barium sulfate; precipitated calcium carbonate; alumina white; magnesium carbonate and white carbon; white pigments such as titanium oxide, white zinc, etc.; black pigments such as carbon black; yellow pigments such as chrome yellow, disazo yellow, Hansa yellow; red pigments such as brilliant carmine 6B, lake red C, permanent red F5R, Rhodamine Lake, etc.; blue pigments such as phthalocyanine blue, Victoria Blue Lake, Prussian Blue; orange pigments such as chrome vermilion, disazo orange; green pigments such as phthalocyanine green, etc.; violet pigments such as methyl violet lake, dioxazine violet, etc.; other pigments such as isoindolinone, benzimidazoline, condensed azo, quinacdrine, etc.; composite oxide pigments; graphite; mica (such as muscovite, phlogopite, synthetic mica, fluorine tetra silicon mica, etc.); metal oxide coated mica (such as titanium oxide coated mica, titanium dioxide coated mica, (hydrated) iron oxide coated mica, mica coated with iron oxides and titanium oxides, mica coated with lower ordered titanium oxides); metal oxide coated graphite (such as titanium dioxide coated graphite, etc.), thin platelet-like alumina; metal oxide coated alumina (such as titanium dioxide coated alumina, iron oxide coated thin platelet-like alumina, Fe₂O₃ coated thin platelet-like alumina, Fe₃O₄ coated thin platelet-like alumina, interference color metal oxide coated thin platelet-like alumina, etc.); MIO; metal oxide coated MIO; metal oxide coated silica flakes and metal oxide coated glass flakes. These inks can be printed on wood, plastic, metal sheet plate, glass, ceramic, paper, corrugated cardboard, film, sheets, canned goods, translucent films of reflectors for LCDs, etc. When the pigments of the present invention are combined with these pigments, new hues, colors and functions can appear. In particular, an appropriate combination with color travel effect pigments may make the pigments according to the present invention also suitable for preventing the counterfeiting of securities, tickets, travel coupons and passenger tickets, etc.

Moreover, when used in printing inks, it is particularly preferred to perform a high plane orientation treatment (mentioned above) to the pigments in the present invention. Pigments subjected to such a surface treatment can be mixed with various kinds of printing inks and used for offset printing, gravure printing, screen printing, ultraviolet cure printing, relief and lithographic printing. The use of pigments that have been subject to a high plane orientation treatment for inks results in improvement of coloration of interference colors on the printed surface.

### Use for Plastics

In the present invention, when incorporated in plastics, the pigments can be mixed with the resin, either directly or after previously forming of pellets, and then incorporated into various types of molded products by means of extrusion molding, calender molding, blow molding, etc. As the resin component, any of polyolefin-based thermoplastic resins and epoxy-based, polyester-based and polyamide (nylon)-based thermosetting resins can be used. A small amount of pigments can be sufficient to effectively produce the color effects of the pigments of the present invention, e.g., when forming a multi-layered plastic bottle, the external appearance of the bottle can be made to appear effectively by incorporating the pigments in the resin of the outer layer. Especially pigments obtained in the present invention on which an additional plane orientation treatment has been performed (as described above) are preferred for the purpose of improving the coloration. Naturally, it is also possible to use the pigments in the present invention on which a weld-line prevention surface treatment (such as encapsulation etc.) has been performed.

The pigments in the present invention can also be used in combination with other pigments. Examples of such pigments include titanium dioxide; calcium carbonate; clay; talc; barium sulfate; white carbon; chromium oxide; zinc oxide; zinc sulfide; zinc powder; metal powder pigments; iron black; yellow iron oxide; red iron oxide; chrome yellow; carbon black; molybdate orange; Prussian Blue; ultramarine blue; cadmium type pigments; fluorescent pigments; soluble azo dyes; insoluble azo dyes; condensed azo dyes; phthalocyanine pigments; condensed polycyclic pigments; composite oxide pigments; graphite; mica (such as, muscovite, phlogopite, synthetic mica, fluorine tetra silicon mica, etc.), metal oxide coated mica (such as titanium oxide coated mica, titanium dioxide coated mica, (hydrated) iron oxide coated mica, mica coated with iron oxides and titanium oxides, mica coated with lower ordered titanium oxides); metal oxide coated graphite (such as titanium dioxide coated graphite, etc.), thin platelet-like alumina; metal oxide coated alumina (such as titanium dioxide coated alumina, iron oxide coated thin platelet-like alumina, Fe₂O₃ coated thin platelet-like alumina, Fe₃O₄ coated thin platelet-like alumina, interference color metal oxide coated thin platelet-like alumina, etc.); MIO; metal oxide coated MIO; metal oxide coated silica flakes and metal oxide coated glass flakes.

### Use for Laser Marking

The pigments in the present invention can be used in various moldings by kneading them into the above-mentioned plastics not only for design purposes but also for facilitating laser printing and enhancing its clarity.

### Use for Cosmetics

The use of the pigments in the present invention for cosmetics includes make-up cosmetics, hair care products, cosmetic packs, etc. The pigments can be used for example in gel, lipstick, foundation (including emulsion, liquid, oil-type emulsions, etc.), cheek rouge, mascara, nail enamel, eyebrow pencil, eye shadow, eye liner, hair products, etc. Those pigments can be used in a proportion of 1-100 wt % based on the formulation. E.g., 1 to 50 wt % for foundations, 1-80 wt % for eye shadow, 1-40 wt % for lipstick and 0.1-20 wt % for nail enamel, can be mentioned.

Examples of the mixing component will be given below. Examples of pigments used in combination with the pigments according to the present invention include titanium dioxide; calcium carbonate; clay; talc; barium sulfate; white carbon; chromium oxide; zinc oxide; zinc sulfide; zinc powder; metal powder pigments; iron black; yellow iron oxide; red iron oxide; chrome yellow; carbon black; molybdate orange; Prussian Blue; ultramarine blue; cadmium type pigments; fluorescent pigments; soluble azo dyes; insoluble azo dyes; condensed azo dyes; phthalocyanine pigments; condensed polycyclic pigments; composite oxide pigments; graphite; metal powder pigments; mica (such as, muscovite, phlogopite, synthetic mica, fluorine tetra silicon mica, etc.), metal oxide coated mica (such as titanium oxide coated mica, titanium dioxide coated mica, (hydrated) iron oxide coated mica, mica coated with iron oxides and titanium oxides, mica coated with lower ordered titanium oxides); metal oxide coated graphite (such as titanium dioxide coated graphite, etc.), thin platelet-like alumina; metal oxide coated alumina (such as titanium dioxide coated alumina, iron oxide coated thin platelet-like alumina, Fe₂O₃ coated thin platelet-like alumina, Fe₃O₄ coated thin platelet-like alumina, interference color metal oxide coated thin platelet-like alumina, etc.); MIO; metal oxide coated MIO; metal oxide coated silica flakes and metal oxide coated glass flakes, sericite, magnesium carbonate, silica, zeolite, hydroxyapatite, chromium oxide, cobalt titanate, glass beads, nylon beads, silicone beads, etc. Examples of organic pigments include red nos. 2, 3, 102, 104, 105, 106, 201, 202, 203, 204, 205, 206, 207, 208, 213, 214, 215, 218, 219, 220, 221, 223, 225, 226, 227, 228, 230-1, 230-2, 231, 232, 405; yellow nos. 4, 5, 201, 202-1, 202-2, 203, 204, 205, 401, 402, 403, 404, 405, 406, 407; green nos. 3, 201, 202, 204, 205, 401, 402; blue nos. 1, 2, 201, 202, 203, 204, 205, 403, 404; orange nos. 201, 203, 204, 205, 206, 207, 401, 402, 403; brown no. 201; violet nos. 201, 401; black no. 401.

Examples of natural colors include salol yellow, carmine, β-carotin, hibiscus color, capsaicin, carminic acid, laccaic acid, gurcumin, riboflavin, shikonin, etc.

Further, examples of other components include oils and fats; surfactants; hydrocarbons such as squalanes, liquid paraffins, palmitic acids, stearic acids, bees wax, myristyl myristate; oil components and other organic solvents such as acetone, toluene, butyl acetate, acetic ester, polyhydric alcohols; waxes; antioxidants; UV absorbers; vitamins; hormones; antiseptic agents; perfumes; etc. By combining the pigments in the present invention with these pigments and components, novel effect colors and functions can be found.

When used in cosmetics, the pigments in the present invention can be used for example in compact cakes, cream, lipstick, etc.; however, they are particularly effective when used in make-up cosmetics, wherein colors are particularly important. Naturally, it is possible to use the pigments in the present invention on which a surface treatment (mentioned above) has been performed beforehand.

### Other Uses

The pigments in the present invention can be used by combining them with color toners for copying machines etc.

Hereinafter, the present invention will be described in more detail by reference to the Example and Comparative Example, which, however, are not intended to limit the present invention.

### Example 1

Preparation of the Colored Interference Pigment Having Metallic Luster with a Reddish Color (Fe₂O₃/SnO₂/[SiO₂/Al(P)])

100 g of a thin platelet-like metal substrate, for example aluminum, with a mass-median-diameter D50 of about 13.5 μm having a layer formed by an anti-corrosive treatment ([SiO₂/Al(P)] obtained according to example 4-b in paragraph of JP, A, 2003-41150) are suspended in 2 liters of water. The suspension is heated to 75° C. under stirring. 186 ml of SnCl₄·5H₂O solution with a concentration of 50 g/l are dropped into the suspension while keeping the pH at 1.8 using 20% by weight aqueous solution of sodium carbonate (preparation of the first layer, “intermediate binder layer”). Then, 4540 g of FeCl₃ (III) aqueous solution with a concentration of 87.75 g/l are dropped until the desired hue of color is reached while keeping the pH at 3.0 using 20 wt % sodium carbonate (preparation of the second layer). From the suspension, the solid parts are filtered, washed, dried, and calcined at 350° C. for 30 minutes, the colored interference pigments having metallic luster with a reddish color are obtained.

### Example 2

Example of Surface Treatments on the Colored Interference Pigment Having Metallic Luster with a Reddish Color (Fe₂O₃/SnO₂/[SiO₂/Al(P)]) According to the Present Invention

10% slurry of Example 1 (Example 1/ion exchanged water in the weight ratio of 100 g/1000 g) is prepared, and temperature is kept at room temperature with stirring. After phosphoric acid (H₃PO₄) solution is added, the slurry is kept it for 30 minutes. Then, the solution of tetraethyl orthosilicate (TEOS) is dropped. After that, the pH of the slurry was adjusted to 6.0, and the temperature is raised to 75° C. Two silane coupling agents, methacryl silane (Z6030: Methacryloxypropyltrimethoxysilane) and epoxy silane (Z6040:3-Glycidoxypropyl Trimethoxysilane) are dropped to the solution. After keeping it for 1 hour, the silane coupling agent of amino silane (Z6020: N-(β-aminoethyl)-γ-aminopropyltrimethoxysilane) solution is added to the solution. After keeping it for 1 hour, the slurry is filtered and washed with ion exchanged water. The cake is dried at 130° C. for 2.5 hours. The dried sample is sieved by 32 μm sieve.

The obtained metal effect pigment comprises 0.025 wt % of P₂O₅, 0.5 wt % of SiO₂, 1.0 wt % of carbon content. All percentages are measured by weight and based on the total weight of metal effect pigment. The carbon content is measured by a CHN analyzer UNICUBE by Elementar.

### Example 3

Example of Surface Treatments on the Colored Interference Pigment Having Metallic Luster with a Reddish Color (Fe₂O₃/SnO₂/[SiO₂/Al(P)]) According to the Present Invention

Meoxal® Victoria Red (Fe₂O₃/SnO₂/[SiO₂/Al(P) of Merck KGaA]) having the mass-median-diameter D50 of about 18 μm, is obtained according to JP 2004004811, JP 2005264144 A, JP3987067 B2. 10% slurry of the above Meoxal® Victoria Red (the above Meoxal® Victoria Red/ion exchanged water in the weight ratio of 100 g/1000 g) is prepared, and temperature is kept at room temperature with stirring. After phosphoric acid (H₃PO₄) solution was added, the slurry is kept it for 30 minutes. Then, the solution of tetraethyl orthosilicate (TEOS) is dropped. After that, the pH of the slurry is adjusted to 6.0, and the temperature is raised to 75° C. Two silane coupling agents, methacryl silane (Z6030: Methacryloxypropyltrimethoxysilane) and epoxy silane (Z6040:3-Glycidoxypropyl Trimethoxysilane) are dropped to the solution. After keeping it for 1 hour, the silane coupling agent of amino silane (Z6020: N-(β-aminoethyl)-y-aminopropyltrimethoxysilane) solution is added to the solution. After keeping it for 1 hour, the slurry is filtered and washed with ion exchanged water. The cake is dried at 130° C. for 2.5 hours.

The dried sample is sieved by 32 μm sieve. The obtained metal effect pigment comprises 0.15 wt % of P₂O₅, 0.5 wt % of SiO₂, 1.8 wt % of carbon content. All percentages are measured by weight and based on the total weight of the metal effect pigment. The carbon content is measured by a CHN analyzer UNICUBE by Elementar.

### Comparative Example 1

Example of Insufficient Surface Treatments on the Colored Interference Pigment Having Metallic Luster with a Reddish Color (Fe₂O₃/SnO₂/[SiO₂/Al(P)])

10% slurry of Example 1 (Example 1/ion exchanged water in the weight ratio of 100 g/1000 g) is prepared and temperature is kept at room temperature with stirring. Then the pH of the slurry is adjusted to 6.0, and the temperature is raised to 75° C. After that, two silane coupling agents, methyacryl silane (Z6030: Methacryloxypropyltrimethoxysilane) and epoxy silane (Z6030: Methacryloxypropyltrimethoxysilane) are dropped to the solution. After keeping it for 1 hour, the silane coupling agent of amino silane (Z6020: N-(β-aminoethyl)-y-aminopropyltrimethoxysilane) solution is added to the solution. After keeping it for 1 hour, the slurry is filtered and washed with ion exchanged water. The cake is dried at 130° C. for 2.5 hours. The dried sample is sieved by 32 μm sieve.

### Comparative Example 2

Example of Insufficient Surface Treatments on the Colored Interference Pigment Having Metallic Luster with a Reddish Color (Fe₂O₃/SnO₂/[SiO₂/Al(P)])

Meoxal® Victoria Red (Fe₂O₃/SnO₂/[SiO₂/Al(P)] of Merck KGaA) having the mass-median-diameter D50 of about 18 μm, is obtained according to JP 2004004811, JP 2005264144 A, JP3987067 B2. 10% slurry of the above Meoxal® Victoria Red (the above Meoxal® Victoria Red/ion exchanged water in the weight ratio of 100 g/1000 g) is prepared, and temperature is kept at room temperature with stirring. Then the pH of the slurry is adjusted to 6.0, and the temperature is raised to 75° C. After that, two silane coupling agents, methyacryl silane (Z6030: Methacryloxypropyltrimethoxysilane) and epoxy silane (Z6030: Methacryloxypropyltrimethoxysilane) are dropped to the solution. After keeping it for 1 hour, the silane coupling agent of amino silane (Z6020: N-(β-aminoethyl)-y-aminopropyltrimethoxysilane) solution is added to the solution. After keeping it for 1 hour, the slurry is filtered and washed with ion exchanged water. The cake is dried at 130° C. for 2.5 hours. The dried sample is sieved by 32 μm sieve.

### Evaluation of the Performance of Humidity Resistance

The metal effect pigments according to the present invention and the metal effect pigments of the comparative examples are investigated by humidity cabinet test as discussed below. The corresponding test results are shown in Table 1.

### Humidity Cabinet Test:

The humidity cabinet test, within HYGROTHERM 519 by ERICHSEN INC, according to DIN EN ISO 6270-2 assesses the behavior of a coating and/or pigments embedded in a coating after storing the coated panels in a chamber for 10 days under 100% humidity and 40° C.

The test is carried out in waterborne coating system. After the end of the humidity cabinet test, the panels are taken out for 1 hour and 4 hours. The performance (humidity resistance) is evaluated through the distinctness of image (DOI) {test scale from 10 (better) to 0 (worse)}.

The results of the humidity cabinet test are shown in Table 1.

TABLE 1
Performance of humidity resistance measured by
humidity cabinet test according to DIN EN ISO
6270-2 (DOI in the humidity cabinet test)

DOI*

| Sample | 1 h | 4 h |
|---|---|---|
| Example 2 | 9 | 9 |
| Example 3 | 9 | 9 |
| Comparative Example 1 | 6 | 6 |
| Comparative Example 2 | 6 | 7 |

*Test scale: from 10 (better) to 0 (worse)

With no surface treatments on the metal effect pigments, e.g., the metal effect pigments obtained from Example 1 shows very poor performance of humidity resistance which can hardly be used for outdoor applications. While with insufficient surface treatments as demonstrated in Comparative Examples 1 and Comparative Examples 2, the resulted metal effect pigments still give out poor performance of humidity resistance (see Table 1). Only with the surface treatments as disclosed in the present application (Example 2 and Example 3 as examples), the resulted metal effect pigments can achieve good performance of humidity resistance (see Table 1).

Hereafter specific examples for the use will be shown. The metal effect pigments with surface-treatments according to the present invention show very good compatibility with the application medium.

### Use Example 1

### Use Examples for Paint:

### Paint Based on Pearlescent Pigments:

| | |
|---|---|
| (Composition A) | |
| Acrydic 47-712 | 70 weight parts |
| Super Beckamine G821-60 | 30 weight parts |
| (Composition B) | |
| Sample of example 2 | 10 weight parts |
| Pearl-luster pigment | 10 weight parts |
| (Composition C) | |
| Ethyl acetate | 50 weight parts |
| Toluene | 30 weight parts |
| n-butanol | 10 weight parts |
| Solvesso #150 | 40 weight parts |

100 weight parts of Composition A are mixed with 20 weight parts of Composition B, the resulting mixture is diluted to obtain a viscosity (12 to 15 seconds with Ford Cup #4) suitable for spray-coating with Composition C, and is spray-coated to form a basecoat layer.

### Clear Paint:

| | |
|---|---|
| Acrydic 44-179 | 14 weight parts |
| Super Beckamine L117-60 | 6 weight parts |
| Toluene | 4 weight parts |
| MIBK (Methyl isobutyl ketone) | 4 weight parts |
| Butyl cellosolve | 3 weight parts |

This composition is coated on the above pearlescent base coating, dried at 40° C. for 30 minutes, air-dried at room temperature and baked (at 130° C. for 30 minutes). The obtained paint film exhibits the interference color having metallic luster with brilliant reddish color having high chromaticity.

### Use Example 2

### Use Example for Plastic:

| | | |
|---|---|---|
| High density polyethylene (pellets) | 100 | weight parts |
| Sample of example 2 | 1 | weight part |
| Magnesium stearate | 0.1 | weight parts |
| Zinc stearate | 0.1 | weight parts |

These components are dry-blended and formed by injection molding. The molding containing a sample of example 2 exhibits the interference color having metallic luster with brilliant reddish color.

### Use Example 3

### Use Example for Ink:

| | |
|---|---|
| CCST medium (nitrocellulose resin) | 10 weight parts |
| Sample of example 2 | 8 weight parts |

The solvent NC 102 is added to the ink composition blended from the above components, and ink with a viscosity of 20 seconds with Zahn Cup No. 3 is prepared. Prints obtained with this ink containing a sample of example 2 exhibits the interference color having metallic luster with brilliant reddish color.

### Use Example 4

### Use Example for Cosmetics

### Use Example for Compact Powder:

| Talc | 50 | weight parts |
|---|---|---|
| Sample of example 2 | 25 | weight parts |
| Color pigments | 5 | weight parts |
| Isopropyl myristate | a suitable amount | |
| Magnesium stearate | 2 | weight parts |

### Formulation for Foundation:

| Talc | 38 | weight parts |
|---|---|---|
| Sample of example 2 | 25 | weight parts |
| Mica (8 μm) | 10 | weight parts |
| Magnesium stearate | 3 | weight parts |
| Nylon powder 12 | 8 | weight parts |
| Yellow iron oxide | 1.9 | weight parts |
| Red iron oxide | 0.8 | weight parts |
| Titanium oxide | 1.0 | weight part |
| Mineral oil (oil component) | a suitable amount | |
| (caprylic acid, capric acid) | 3.3 | weight parts |
| triglyceride (oil component) | | |
| Butylparaben | 0.1 | weight parts |

### INDUSTRIAL APPLICABILITY

The metal effect pigment of the present invention is one in which a thin platelet-like metal substrate is coated with one or more layers of metal compounds selected from metal oxides, metal oxide hydrates, metal suboxides, metals, metal fluorides, metal sulfides, metal carbides, metal nitrides, metal oxynitrides, and mixtures thereof. Based on the metal effect pigment, the surface treatments comprising phosphoric acid (H₃PO₄), and tetraethyl orthosilicate (TEOS) and at least one organic coupling agent to the substrate are performed. The metal effect pigments with the surface-treatments show excellent humidity resistance performance without any influence on the chroma, luster and color of base metal effect pigments. Therefore, it can be used in paints, coated films, automotive coatings, industrial coatings, pigment preparations, pigment pastes, radar transparent coatings, lidar applications, painted materials, inks, printed materials, plastics, moldings, laser marking, cosmetics, and others.

1. A metal effect pigments, having a surface treatment with A) phosphoric acid (H₃PO₄), B) tetraethyl orthosilicate (TEOS) and C) at least one organic coupling agent.

2. The metal effect pigments according to claim 1, having a surface treatment with three organic coupling agents are used.

3. The metal effect pigments according to claim 1, wherein the at least one organic coupling agent is selected from alkyl silanes, epoxy silanes, (meth)acryl silanes, amino silanes, and vinyl silanes.

4. The metal effect pigments according to claim 1, wherein the amount of C) the at least one organic coupling agent/agents is in the range of 0.3-3.0% by weight calculated as carbon content based on the total weight of the metal effect pigments.

5. The metal effect pigments according to claim 1, wherein the amount of A) phosphoric acid compounds (H₃PO₄) is in the range of 0.01-1.0% by weight calculated as P₂O₅ based on the total weight of the metal effect pigments.

6. The metal effect pigments according to claim 1, wherein the amount of B) tetraethyl orthosilicate (TEOS) is in the range of 0.1-1.5% by weight calculated as SiO₂ based on the total weight of the metal effect pigments.

7. The metal effect pigments according to claim 1, wherein the metal effect pigments comprises a thin platelet-like metal substrate, coated with one or more layers of metal compounds selected from metal oxides, metal oxide hydrates, metal suboxides, metals, metal fluorides, metal sulfides, metal carbides, metal nitrides, metal oxynitrides, and mixtures thereof, and wherein the thin platelet-like metal substrate is selected from at least one type of metal or metal alloy selected from aluminum, titanium, gold, silver, iron, stainless steel, copper, zinc, tin, nickel, and chromium.

8. The metal effect pigments according to claim 7, wherein the thin platelet-like metal substrate is coated with one or more metal oxides of one or more metals and/or metal sulfides selected from the group consisting of iron, titanium, aluminum, zirconium, tin, zinc, bismuth, calcium, manganese, cerium, chromium, cobalt, silicon, and boron.

9. The metal effect pigments according to claim 7, wherein the metal effect pigments further comprises a layer obtained from the following steps: treating the surface of the thin platelet-like metal substrate with phosphoric acids compounds and/or boric acids compounds, followed by coating with one or more hydrated metal oxide layers of one or more metals selected from the group consisting of silicon, aluminum, zirconium, and titanium by a sol-gel method.

10. A method for preparing a metal effect pigments according to claim 1, comprising the following steps:
Dispersing/suspending the metal effect pigments in water and/or one or more solvents,
Performing the surface treatment by adding A) phosphoric acid (H₃PO₄), B) tetraethyl orthosilicate (TEOS) and C) at least one organic coupling agent into the water and/or the solvent(s), and
Drying the metal effect pigments.

11. The method for preparing a metal effect pigments according to claim 10, wherein the surface treatment can be performed by the wet-chemical methods and/or by the sol-gel process.

12. A waterborne coating system, comprising the metal effect pigments according to claim 1.

13. A paints, coated films, automotive coatings, industrial coatings, pigment preparations, pigment pastes, radar transparent coatings, lidar applications, painted materials, inks, printed materials, plastics, moldings, laser marking, or cosmetics comprising the effect pigment of claim 1.
